(19) United States
(12) Patent Application Publication
(10) Pub. No.: US 2011/0293757 A1
(43) Pub. Date: Dec. 1, 2011

(54) Compositions Comprising Tea Tree Oil and Methods for the Prevention and Treatment of Cancer

(75) Inventors: Thomas V. Riley, East Perth (AU); Sara J. Greay, Karrinyup (AU); Christine F. Carson, Hilton (AU); Manfred W. Beilharz, Nedlands (AU); Demelza J. Ireland, Ashfield (AU); Haydn T. Kissick, Subiaco (AU)

(73) Assignee: Novasel Australia Pty. Ltd., Mudgeeraba (AU)

(21) Appl. No.: 13/059,828

(22) PCT Filed: Jul. 17, 2009
(86) PCT No.: PCT/AU2009/000914

§ 371 Date: Aug. 16, 2011

(30) Foreign Application Priority Data
Jul. 18, 2008 (AU) 2008903693
Oct. 7, 2008 (AU) 2008905198

(51) Int. Cl. A61K 36/61; C07C 13/47; C07C 35/37; A61P 35/00; C07D 493/08; C07C 35/18

(52) U.S. Cl. 424/769; 549/397; 568/827; 568/817; 585/22

(57) ABSTRACT

Compositions for the treatment and/or prevention of cancer are described. Preferably the composition comprises a therapeutically effective amount of tea tree oil from Melaleuca alternifolia, together with a pharmaceutically or therapeutically acceptable carrier and/or diluent. Methods for the treatment and/or prevention of cancer are also described. Preferably the method is used for the treatment and prevention of skin cancer. More preferably, the method is used for the treatment and prevention of basal cell carcinoma, squamous cell carcinoma and/or melanoma.

### FIELD OF THE INVENTION

This invention relates to compositions and methods for the prevention and treatment of cancer. In particular, it provides compositions useful for the topical application of tea tree oil together with a suitable pharmaceutically or therapeutically acceptable carrier and/or diluent for the prevention and/or treatment of skin and subcutaneous cancers. It also provides a method for the prevention and/or treatment of skin and subcutaneous cancers comprising administering to a subject in need thereof, a therapeutically acceptable amount of tea tree oil, together with a suitable pharmaceutically or therapeutically acceptable carrier and/or diluent.

### BACKGROUND ART

In spite of numerous advances in medical research, cancer remains one of the leading causes of death. Traditional modes of clinical care, such as surgical resection, radiotherapy and chemotherapy, have a significant failure rate, especially for solid tumours. Failure occurs either because the initial tumour is unresponsive, or because of recurrence due to regrowth at the original site or metastasis. Cancer and its prevention remains a central focus for medical research and development.

In Australia, 83% of all cancers diagnosed are skin cancers, the highest rate worldwide (Australian Institute of Health and Welfare (AIHW) & Australasian Association of Cancer Registries (AACR) 2004). Moreover, Australia has the second highest mortality rate of malignant melanoma (Geller, 2007).

The most common type of skin cancer is basal cell carcinoma which accounts for about 75% of all skin cancers. Squamous cell carcinoma accounts for 20% and melanoma accounts for less than 5% of all skin cancers. Disturbingly, the incidence of melanoma is increasing (1000 cases annually in Western Australia (WA) alone) and while the mortality rate for non-melanoma skin cancers is low, morbidity and medical costs of treatment are high. Furthermore, the incidence rates for all skin cancers are increasing.

The two in vivo cancer models that have been used to obtain data are murine melanoma and mesothelioma. Both are well characterised mouse models of tumour growth in immuno-competent animals.

Tumours may develop through immune evasion mechanisms which include reduced antigenicity, development of a local immunosuppressive environment, deletion and anergy of tumour specific cytotoxic cells, amongst many others. In general, immunotherapy for the treatment of cancer aims to boost the immune response mounted against the tumour to reverse these mechanisms of immune evasion developed by the tumour (Rabinovich, 2007).

Imiquimod, a successfully used topical skin cancer treatment, initiates it's mechanisms of action by activation of Nuclear Factor-κB (NF-κB) stimulating pro-inflammatory cytokines, by apoptosis and by dendritic cell (DC) activation (Lee 2007). It's topical application is associated with redness, drying and scabbing. DCs are responsible for priming T-cell mediated clearance of tumour cells; and their activation represents a promising approach for treatment of immunogenic tumours.

The antitumour topical diterpene agent 3-Ingenyl-Angelate (also known as ingenol mebutate and PEP005) from Euphorbia peplus has been used for treatment of murine subcutaneous tumours of B16 melanoma, UV induced squamous carcinoma and Lewis lung carcinoma. 18 μg of the component induces tumour regression with just 3 topical treatments (Ogbourne, 2004) and similarly to imiquimod, the treatment with diterpene agent 3-Ingenyl-Angelate elicits skin irritation. This is believed necessary for the antitumour effect. 3-Ingenyl-Angelate's antitumour mode of action involves necrotic cell death (Ogbourne, 2004) and the migration of neutrophils (Challacombe, 2006) associated with an inflammatory response. This effect is comparable to that observed following a tea tree oil (abbreviated hereafter as “TTO”) treatment regime.

The skin inflammation observed following the aforementioned topical treatments is associated with the activation of DCs and migration of neutrophils to the localised area of treatment. The skin inflammation observed following the aforementioned topical treatments and that observed with topical TTO treatment, supports the hypothesis that TTO results in localised activation of the immune response generated towards a tumour could involve the activation of DCs and migration of neutrophils and that this observation forms the basis to develop an immunotherapeutic treatment for skin cancer.

The inventors have surprisingly found that that the administration of TTO can prevent and treat the onset of cancer, including subcutaneous cancers. Regression of established tumours and cessation of tumour growth have also been demonstrated following administration of TTO.

TTO, which is obtained from distillation of the leaves of Melaleuca alternifolia, is known as a natural preservative, having antimicrobial properties. It has been demonstrated that TTO is effective against Streptococci when used topically as a wound disinfectant (Carson, 1996). Over 90 chemical components have been isolated from TTO, and TTO consists of a mixture of monoterpenes, sesquiterpenes and terpene alcohols. The antimicrobial activity that has been demonstrated with TTO is mainly due to terpinen-4-ol but other components have activity by themselves or may contribute synergistically. The ISO Standard specifies a minimum of 30% terpinen-4-ol and a maximum of 15% 1,8-cineole.

### SUMMARY OF THE INVENTION

According to the present invention, there is provided a composition for the prevention of cancer, wherein the composition comprises a therapeutically effective amount of TTO.

According to a second embodiment, the invention provides a composition for the treatment of cancer, wherein the composition comprises a therapeutically effective amount of TTO.

According to a third embodiment, the invention provides a method for the prevention of cancer, wherein the method comprises administering to a subject in need thereof, a composition comprising a therapeutically effective amount of TTO.

According to a forth embodiment, the invention provides a method for the treatment of cancer, wherein the method comprises administering to a subject in need thereof, a composition comprising a therapeutically effective amount of TTO.

Preferably, the cancer is a cancer selected from the group consisting of sarcomas, carcinomas and other solid tumour cancers, including, but not limited to germ line tumours, tumours of the central nervous system, breast cancer, prostate cancer, skin cancer, cervical cancer, uterine cancer, lung cancer, ovarian cancer, testicular cancer, thyroid cancer, astrocytoma, glioma, pancreatic cancer, stomach cancer, liver cancer, colon cancer, renal cancer, bladder cancer, oesophageal cancer, cancer of the larynx, cancer of the parotid, cancer of the biliary tract, rectal cancer, endometrial cancer, adenocarcinomas, small cell carcinomas, neuroblastomas, mesotheliomas, adrenocortical carcinomas, epithelial carcinomas, desmoid tumours, desmoplastic small round cell tumours, endocrine tumours, Ewing sarcoma family tumours, germ cell tumours, hepatoblastomas, hepatocellular carcinomas, lymphomas, non-rhabdomyosarcome soft tissue sarcomas, osteosarcomas, peripheral primative neuroectodermal tumours, retinoblastomas, rhabdomyosarcomas and Wilms tumours.

More preferably, the cancer is skin cancer. Even more preferably the skin cancer is selected from the group consisting of basal cell carcinoma cell carcinoma, squamous cell carcinoma and melanoma.

Alternatively the cancer is a cancer of a mucosal surface.

According to a fifth embodiment, the invention provides a dosage form, comprising a therapeutically effective amount of a composition according to this invention, stored in a vial or container, wherein the vial or container is labelled with instructions that the composition is administered topically to the subject's skin for the treatment or prevention of cancer. Preferably the container or vial is sealed. Preferably, the cancer is skin cancer.

Preferably, the TTO is derived from Melaleuca alternifolia. Preferably the TTO is compliant with the International Standard 4730 (Standardisation, 2004) and contains a minimum of 30% terpinen-4-ol and a maximum of 15% 1,8-cineole.

In one aspect the subject has not been diagnosed with cancer but may be at risk of developing cancer. For example, the subject may suffer with precancerous skin lesions also known as actinic or solar keratosis and may wish to treat actinic keratosis and/or prevent the onset of skin cancer.

Preferably, the subject in need of such treatment or prevention is a human.

Preferably, the composition comprises a pharmaceutically or therapeutically acceptable carrier and/or diluent. For example the composition comprises conventional solvents, dispersion media, fillers, aqueous solutions, antibacterial and antifungal agents and/or absorption-promoting agents. For example the composition comprises dimethyl sulphoxide (DMSO).

Preferably, the composition is in a form selected from the group consisting of cosmetically acceptable liquids, creams, oils, lotions, ointments, gels, roll-on liquids, skin patches, sprays, glass bead dressings, synthetic polymer dressings impregnated with basic milk factors, solids, conventional cosmetic night creams, foundation creams, suntan lotions, hand lotions, insect repellents, make-up, make-up bases and masks.

In one preferred embodiment, the composition is regulated as a prescription pharmaceutical.

In another preferred embodiment, the composition is a non prescription, “over the counter” medicine.

In another preferred embodiment, the composition is a personal care product. For example the composition is a cosmetic or health product.

Preferably, the composition is administered intravenously, intra-arterially, intraperitoneally, intramuscularly, subcutaneously, intranasally or transdermally.

Preferably, the composition is adapted for topical administration. More preferably the composition comprises an agent to improve the trans-dermal delivery of the TTO. For example, the composition comprises a surfactant. In another example, the composition comprises DMSO. In another example, the composition is an emulsion. In another example, the composition is a microemulsion. In another example, the composition is a nanoemulsion.

Preferably, the composition is administered to the subject topically. For example, the TTO is formulated in a composition adapted for topical application and is applied to the skin. Preferably the composition is administered at least once daily.

Preferably, the composition comprises between 0.1% and 80% TTO by weight of the composition. More preferably, the composition comprises between 1% and 30% TTO by weight of the composition. Most preferably, the composition comprises between 3% and 15% TTO by weight of the composition. For example, the composition comprises a percentage of TTO by weight of composition of any one of the following: 15%, 14%, 13%, 12%, 11%, 10%, 9%, 8%, 7%, 6%, 5%, 4%, 3%.

Preferably, when the composition is administered topically it is administered once daily. For example, a composition comprising 3% TTO by volume and DMSO is administered topically once daily. Alternatively, a composition comprising 3% TTO by volume and a suitable gel is administered topically once daily. Alternatively, composition comprising 10% TTO by volume and DMSO is administered topically once daily or once every two days. Alternatively, composition comprising 5% TTO by volume and DMSO is administered topically once daily. Preferably, the quantity of the composition that is applied to the skin is an amount effective to treat the cancer or prevent the onset of the cancer.

Preferably, the composition comprises DMSO at a percentage concentration between 1 and 90% by weight.

Preferably, the composition comprises DMSO at a percentage concentration selected from the group consisting of: 3% TTO and 97% DMSO by weight; 4% TTO and 96% DMSO by weight; 5% TTO and 95% DMSO by weight; 6% TTO and 94% DMSO by weight; 7% TTO and 93% DMSO by weight; 8% TTO and 92% DMSO by weight; 9% TTO and 91% DMSO by weight; 10% TTO and 90% DMSO by weight. Most preferably, the composition comprises a quantity of DMSO in an amount effective when combined with TTO to treat or prevent the onset of cancer.

In another preferred embodiment, the invention comprises a combination of TTO constituents in a TTO-like solution, such as: 40% terpinen-4-ol, 20% γ-terpinene, 10% α-terpinene 5% 1,8-cineole and 5% p-cymene, 20% Ethanol (EtOH) included to total the solution composition to 100%. For example a composition comprising 10% TTO-like solution by volume and DMSO is administered topically once daily.

Preferably, the composition adapted for the treatment and/or prevention of cancer, comprises a therapeutically effective amount of terpinen-4-ol.

Preferably, the composition adapted for the treatment and/or prevention of cancer, comprises a therapeutically effective amount of terpinen-4-ol, γ-terpinene, α-terpinene 1,8-cineole and p-cymene. Preferably, the composition comprises 40% terpinen-4-ol, 20% γ-terpinene, 10% α-terpinene 5% 1,8-cineole, 5% p-cymene and 20% ethanol.

Alternatively, the composition comprises between 0.1% and 80% TTO-like solution by weight of the composition. More preferably, the composition comprises between 1% and 30% TTO-like solution by weight of the composition. Most preferably, the composition comprises between 3% and 15% TTO-like solution by weight of the composition. For example, the composition comprises a percentage of TTO-like solution by weight of composition of any one of the following: 15%, 14%, 13%, 12%, 11%, 10%, 9%, 8%, 7%, 6%, 5%, 4%, 3%.

In another preferred embodiment, the invention comprises a combination of any TTO constituents.

In another preferred embodiment, the composition further comprises a second agent for the treatment and/or prevent of cancer. For example, the composition may comprise a chemotherapy agent, an antibody or immunomodulatory agent directed at the cancerous cells. By way of illustration, the invention may comprise Imiquimod, 5-Fluorouracil, 1-isobutyl-1H-imidazo[4,5-4-quinolin-4-amine,3-ingenyl angelate or perillyl alcohol.

In another preferred embodiment, the invention comprises the use of a composition according to this invention in the manufacture of a medicament for the treatment or prevention of cancer, in accordance with the methods of the invention.

### BRIEF DESCRIPTION OF THE FIGURES

FIGS. 1A and B: These figures represent the change in mesothelioma (AE17) and melanoma (B16) tumour area following 10% TTO in DMSO treatment. C57BL/6J mice with subcutaneous tumours (A) (˜9 mm² AE17) and (B) (˜9 mm² B16) were treated topically daily for 4 days with 50 μl DMSO (control), 50 μl 10% TTO in DMSO (n=15 for all groups±SEM).

FIGS. 2A and B: These figures represent the change in mesothelioma (AE17) and melanoma (B16) tumour area following treatment with 3% TTO in DMSO or 3% Novasel® Gel. C57BL/6J mice with subcutaneous tumours (A) (˜9 mm² AE17) and (B) (0 mm² B16) were treated topically daily for 16 days and 13 days respectively with 50 μl DMSO solvent control or 50 μl of 3% TTO in DMSO or 50 μl of 3% TTO Novasel® Gel (n=3 for all groups±SEM).

FIGS. 3A and B: These figures represent the change in mesothelioma tumour area following treatment with 5% (daily and twice daily) and 10% TTO in DMSO (every second day and daily). C57BL/6J mice with established (˜9 mm²) AE17 mesothelioma subcutaneous tumours were treated topically with: (A) 50 μl DMSO solvent control, 50 μl of 5% TTO in DMSO daily (for 8 days), 50 μl of 10% TTO in DMSO every second day (for 4 days) and (B) 50 μl DMSO solvent control, 50 μl of 5% TTO in DMSO twice daily (for 4 days) and 50 μl of 10% TTO in DMSO daily for 4 days (n=3 for all groups±SEM).

FIGS. 4A and B: These figures represent the requirement of the inclusion of DMSO with topical TTO. C57BU6J mice with subcutaneous tumours (˜9 mm² AE17) treated topically with (A) 5 μl of neat TTO (n=16) compared with untreated controls (n=12). Data are represented as the mean±SEM of 4 independent experiments. B: Topical treatment with 50 μl 10% TTO in isopropanol (n=7) or 10% TTO in acetone (n=8) compared to untreated controls (n=8). Data are the mean±SEM.

FIG. 5: This figure represents the effect 5 major components of TTO in combination with each other in a “TTO-like” formulation with DMSO. C57BL/6J mice with subcutaneous tumours (˜9 mm² AE17) treated topically with 50 μl 10% TTO-like (n=15) compared with untreated controls (n=15). Data are represented as the mean±SEM of 3 independent experiments.

FIG. 6: This figure represents the lack of efficacy 5 major components of TTO when applied singly with DMSO. C57BU6J mice with subcutaneous tumours (˜9 mm² AE17) treated topically with 50 ul 4% terpinen-4-ol, 2% γ-terpinene, 1% α-terpinene, 0.5% 1,8-cineole and 0.5% p-cymene, (n=3) compared with untreated controls (n=3).

FIG. 7: This figure represents the effect of 10% topical TTO/DMSO C57BL6/J skin by H&E staining of histology sections through the skin of C57BL/6J mice bearing AE17 mesothelioma tumours (A) Day 0, prior to treatment (B) 1 day post treatment with 90% DMSO alone (C) 1 day post treatment with 10% TTO/DMSO (D) post 3 treatment with 90% DMSO (E) post 3 treatments with 10% TTO/DMSO.

### DETAILED DESCRIPTION OF THE INVENTION

General

Those skilled in the art will appreciate that the invention described herein is susceptible to variations and modifications other than those specifically described. It is to be understood that the invention includes all such variation and modifications. The invention also includes all of the steps, features, compositions and compounds referred to or indicated in the specification, individually or collectively and any and all combinations or any two or more of the steps or features.

The present invention is not to be limited in scope by the specific embodiments described herein, which are intended for the purpose of exemplification only. Functionally equivalent products, compositions and methods are clearly within the scope of the invention as described herein.

The entire disclosures of all publications (including patents, patent applications, journal articles, laboratory manuals, books, or other documents) cited herein are hereby incorporated by reference. No admission is made that any of the references constitute prior art or are part of the common general knowledge of those working in the field to which this invention relates.

Throughout this specification, unless the context requires otherwise, the word “comprise”, or variations such as “comprises” or “comprising”, will be understood to imply the inclusion of a stated integer or group of integers but not the exclusion of any other integer or group of integers.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

### DESCRIPTION OF THE PREFERRED EMBODIMENTS

The term ‘tea tree oil’ (abbreviated as “TTO”) refers to the substance which is obtained from the distillation of the leaves or other biomass, including terminal branches, of Melaleuca alternifolia; this distillation is condensed following which the clear pale yellow oil is separated from the aqueous distillate. This yields oil of approximately 1-2% of wet plant material (reviewed in (Carson, 2006). The composition of the oil is regulated by an international standard for “Oil of Melaleuca-terpinen-4-ol type”. This states the 14 major components of TTO must be present at specific minimum or maximum concentrations (Standardisation, 2004). TTO must contains a minimum of 30% terpinen-4-ol and a maximum of 15% 1,8-cineole.

The term “cancer” include all cancers, including sarcomas, carcinomas and other solid tumour cancers, including, but not limited to germ line tumours, tumours of the central nervous system, breast cancer, prostate cancer, skin cancer (including basal cell carcinoma cell carcinoma, squamous cell carcinoma and melanoma), cervical cancer, uterine cancer, lung cancer, ovarian cancer, testicular cancer, thyroid cancer, astrocytoma, glioma, pancreatic cancer, stomach cancer, liver cancer, colon cancer, renal cancer, bladder cancer, oesophageal cancer, cancer of the larynx, cancer of the parotid, cancer of the biliary tract, rectal cancer, endometrial cancer, adenocarcinomas, small cell carcinomas, neuroblastomas, mesotheliomas, adrenocortical carcinomas, epithelial carcinomas, desmoid tumours, desmoplastic small round cell tumours, endocrine tumours, Ewing sarcoma family tumours, germ cell tumours, hepatoblastomas, hepatocellular carcinomas, lymphomas, non-rhabdomyosarcome soft tissue sarcomas, osteosarcomas, peripheral primitive neuroectodermal tumours, retinoblastomas, rhabdomyosarcomas, Wilms tumours, and the like.

The term “subject” as used herein refers to any animal having cancer which requires treatment or who desires to prevent the onset of cancer. The subject may be a human, or may be a domestic or companion animal. While it is particularly contemplated that the compounds of the invention are suitable for use in medical treatment of humans, it is also applicable to veterinary treatment, including treatment of companion animals such as dogs and cats, and domestic animals such as horses, cattle and sheep, or zoo animals such as non-human primates, felids, canids, bovids, and ungulates.

Generally, the terms “treating”, “treatment” and the like are used herein to mean affecting a subject, tissue or cell to obtain a desired pharmacological and/or physiological effect. The effect may be therapeutic in terms of a partial or complete cure of the cancer. “Treating” as used herein covers any treatment of cancer in a subject; inhibiting the cancer, i.e. arresting its development; or relieving or ameliorating the effects of the cancer, i.e., cause regression of the effects of the cancer. “Preventing” or “prevention” as used herein covers any prevention of cancer and includes any partial or complete prevention of cancer or its symptoms in a subject who does not wish to have the disease, but has not yet been diagnosed as having it.

As used herein, the term “therapeutically effective amount” means an amount of a compound of the present invention effective to yield a desired therapeutic response, for example to prevent or treat cancer. The specific “therapeutically effective amount” will of course vary with such factors as the particular condition being treated, the physical condition and clinical history of the subject, the type of animal being treated, the duration of the treatment, the nature of concurrent therapy (if any), and the specific formulations employed and the structure of the composition. The concentration of TTO in the composition is not critical, but should be an amount effective to treat cancer or to prevent or delay the onset of cancer. The concentration of TTO employed can be determined empirically, on the basis of the response of cells in vitro and response of experimental animals to the TTO or formulations containing TTO. Suitable methods to determine therapeutically effective amounts are described in Example 2. The amount of active ingredient which may be combined with the carrier materials to produce a single dosage will vary, depending upon the host to be treated and the particular mode of administration.

Preferentially the amount by weight of the TTO used in the composition will comprise from about 0.1% to about 80% by weight of the composition, more preferably 1% to 30% by weight of the composition, with a range of 3% to 15% by weight of the composition being highly desirable. In an illustration of the invention, the TTO will constitute about 10% of the total weight of the composition.

Administration may be intravenous, intra-arterial, intraperitoneal, intramuscular, subcutaneous, intracavity, intranasal or transdermal. For in vitro studies the agents may be added or dissolved in an appropriate biologically acceptable buffer and added to cells or tissue. Preferably, the composition is administered topically to the skin or mucosal surface.

Methods and carriers for the preparation of pharmaceutical and therapeutic compositions are well known in the art, as set out in textbooks such as Remington's Pharmaceutical Sciences,18th Edition, Mack Publishing Company, Easton, Pa., USA, the contents of which is incorporated herein. Suitable pharmaceutically or therapeutically acceptable carriers and/or diluents include conventional solvents, dispersion media, fillers, aqueous solutions, antibacterial and antifungal agents, absorption-promoting agents, and the like. Frequently used carriers and/or diluents include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol and polyhydric alcohols. Intravenous vehicles include fluid and nutrient replenishers. Preservatives include antimicrobial, anti-oxidants, chelating agents and inert gases.

The pH and exact concentration of the various components of the composition are adjusted according to routine skills in the art. See Goodman and Gilman's The Pharmacological Basis for Therapeutics (7th ed., 1985).

The compositions are preferably prepared and administered in dosage units. For treatment of a subject, depending on activity of the compound, manner of administration, nature and severity of the disorder, age and body weight of the subject, different daily doses can be used. Under certain circumstances, however, higher or lower daily doses may be appropriate. The administration of the daily dose can be carried out both by single administration in the form of an individual dose unit or else several smaller dose units and also by multiple administration of subdivided doses at specific intervals. The compositions according to the invention may be administered locally or systemically in a therapeutically effective dose. Amounts effective for this use will, of course, depend on the severity of the disease and the weight and general state of the subject. Typically, dosages used in vitro may provide useful guidance in the amounts useful for in situ administration of the pharmaceutical composition, and animal models may be used to determine effective dosages for treatment of the cytotoxic side effects.

Aqueous suspensions normally contain the active materials in admixture with excipients suitable for the manufacture of aqueous suspensions. Such excipients may be suspending agents such as sodium carboxymethyl cellulose, methyl cellulose, hydroxypropylmethylcellulose, sodium alginate, polyvinylpyrrolidone, gum tragacanth and gum acacia; dispersing or wetting agents, which may be (a) a naturally occurring phosphatide such as lecithin; (b) a condensation product of an alkylene oxide with a fatty acid, for example, polyoxyethylene stearate; (c) a condensation product of ethylene oxide with a long chain aliphatic alcohol, for example, heptadecaethylenoxycetanol; (d) a condensation product of ethylene oxide with a partial ester derived from a fatty acid and hexitol such as polyoxyethylene sorbitol monooleate, or (e) a condensation product of ethylene oxide with a partial ester derived from fatty acids and hexitol anhydrides, for example polyoxyethylene sorbitan monooleate.

The composition may be adapted for topical application and may be in a form selected from the group comprising cosmetically acceptable liquids, creams, oils, lotions, ointments, gels, roll-on liquids, skin patches, sprays, glass bead dressings, synthetic polymer dressings impregnated with basic milk factors, solids, conventional cosmetic night creams, foundation creams, suntan lotions, hand lotions, insect repellents, make-up, make-up bases and masks. Except insofar as any conventional medium or agent is incompatible with the active ingredient, use thereof in the cosmetic compositions of the present invention is contemplated. Compositions of the present invention adapted for topical delivery will desirably possess bioadhesive or mucoadhesive properties. Suitable vehicles for topical administration of TTO to the skin or mucosal surface include: DMSO (dimethyl sulfoxide), ethanol, acetone, phosphatidyl choline and isopropanol gels. In one example, the composition may be in the form of:
(i) a spray, comprising TTO together with ethanol or prophylene glycol;
(ii) a lotion, comprising TTO together with any one of the following: cetomacrogol lotion, aminobenzoic acid lotion or alcohol;
(iii) a gel, comprising TTO together with any one of the following: poloxamer gel 8E, poloxamer gel 8C, chlorhexidine gel or Lutrol® F127; or
(iv) an ointment, comprising TTO together with any one of the following: liquid paraffin or white soft paraffin; or
(v) a buffered cream, comprising TTO together with any one of the following: emulsifying ointment, glycerol, cetomacrogol emulsifying wax 15 or propylene glycol.

In a preferred form, the composition is adapted for topical application to the skin. Preferably, the composition comprises an agent to promote absorption into the skin. Preferably, the composition has bioadhesive properties.

In another preferred form, the composition is adapted for topical application to a mucosal surface and in particular is adapted for oesophageal, oral cavity, vaginal, rectal and buccal applications. Preferably, the composition has bioadhesive and mucoadhesive properties.

In another preferred embodiment, the composition adapted for topical application is adapted for personal care applications. For example, the composition adapted for personal care applications is used by subjects who have cancer and wish to treat it or who do not have cancer but wish to prevent the cancers occurrence. In particular, the subject wishes: to treat cancer or prevent the occurrence of skin cancer and the TTO is formulated into a composition adapted for cosmetic skin care preparations, such as sun burn creams, gels lotions, makeup preparations. In a preferred form the composition adapted for personal care applications is a cosmetic. For example, the composition has two properties: (1) for the treatment or prevention of cancer; and (2) a cosmetic property. The cosmetic composition may be in any form. Suitable forms include but are not limited to lotions, creams, sticks, roll-ons formulations, mousses, aerosol sprays, pad-applied formulations, and film-forming formulations.

Preparation of the above-named cosmetic compositions and others may be accomplished with reference to any of the cosmetic formulation guidebooks and industry journals which are available in the cosmetic industry. These references supply standard formulations which may be modified by the addition or substitution of the TTO of the present invention into the formulation. Suitable guidebooks include Cosmetics and Toiletries Magazine, Vol. 111 (March, 1996); Formulary: Ideas for Personal Care; Croda, Inc, Parsippany, N.J. (1993); and Cosmeticon: Cosmetic Formulary, BASF, which are hereby incorporated in their entirety by reference.

In a further embodiment, the composition adapted for topical application is an emulsion, microemulsion or nanoemulsion. Most preferably, the composition adapted for topical application is a microemulsion. Most preferably, the microemulsion composition will exist as a gel or will be a liquid that is capable of gelatinising upon contact with dermal or mucosal tissue.

For example, the microemulsions as described herein will comprise an amount by weight of a block copolymer of about 10% to about 50% by weight, more preferably the amount by weight block copolymer will be between about 10.1% and 40% by weight of the emulsion while an amount by weight of the block copolymer between any of the following ranges will be highly desirable: 10.5% to 35%, 11% to 30%, 12% to 25%, 13% to 20% or 14% to 18% by weight of the emulsion. Thus, as an illustration of the invention, the block copolymer may comprise 15% by weight of the emulsion.

In a preferred embodiment of the invention, the microemulsion or composition will possess bioadhesive or mucoadhesive properties. Such properties will be consistent with the microemulsion or composition being prepared in either a liquid or more preferably a gel form. When prepared in this manner the microemulsion or composition will be useful for topical and/or mucosal application of water insoluble or sparingly soluble active agents to oesophageal, otic, vaginal, rectal or ophthalmic surfaces, or for application to the epidermis of an animal (such as skin in human). Desirably, the microemulsion or composition will either exist as a gel or will be prepared in such a manner that it is capable of gelatinising upon contact with dermal or mucosal tissue.

When preparing a microemulsion in accordance with the first embodiment of the invention, ideally, the TTO and a thermo-reversible copolymer will be mixed at a cold temperature. When this is done at a cold temperature at the weight ranges specified herein the composition forms a stable microemulsion capable of application to dermal or mucosal tissue.

The copolymer for use in the present invention is preferably a block copolymer of ethylene oxide and propylene oxide (poloxamer) preferably those represented by the formula:

HO(C₂H₄O)ₐ(C₃H₆O)_{b}(C₂H₄O)ₐH

Where ‘b’ is between 15 and 67 and ‘a’ is between 2 and 130, and the total proportion of ‘a’ units amounts to from 20% to 90% by weight of the poloxamer. The molecular weight of the poloxamer ranges from preferably about 1,000 to 20,000 and it will preferentially have thermo-reversible properties. By way of example only the block copolymer may be poloxamer 407, such as that sold as Pluronic® F127 (BASF Corporation) or Synperonic PE/F127 (Uniqema). According to the invention the preferred emulsifier is a fatty acid component with a polyethoxylated side chain. For example, suitable emulsifiers might be Laureth-4, Laureth-23, PPG-26-Buteth-26/PEG-40 Hydrogenated castor oil or PEG-40 Hydrogenated castor oil. When such emulsifiers are used in the invention the amount by weight of the emulsifier will vary generally from about 0.5% to about 50% by weight of the microemulsion. This particular composition is well-suited for transdermal or transmucosal delivery of TTO. For example, the composition is applied to the skin or mucosal surface for the prevention or treatment of a cancer.

When prepared according to the method of the invention, the microemulsion composition comprising the TTO can further include one or more pharmaceutically acceptable additives, excipients carriers and diluents. Such additives, excipients carriers and diluents include, without limitation, water, saline, ethanol, dextrose, glycerol, lactose, dextrose, sucrose sorbitol, mannitol, starches, gum acacia, calcium phosphates, alginate, tragacanth, gelatine, calcium silicate, microcrystalline cellulose, polyvinylpyrrolidone, cellulose, water syrup, methyl cellulose, methyl and propylhydroxybenzoates, talc, magnesium stearate and mineral oil or combinations thereof. The formulations can additionally include lubricating agents, pH buffering agents, wetting agents, emulsifying and suspending agents, preserving agents, sweetening agents or flavouring agents, antifoaming agents, polymers, antioxidants, chelating agents, viscomodulators, tonicifiers, flavorants, colorants, odorants, opacifiers, suspending agents, binders, fillers, plasticizers, lubricants, and mixtures thereof. The particular selection of constituent that can be included in the compositions described herein will generally depend on the type of preparation.

In addition, an acid or a base may be incorporated into the microemulsion composition comprising the TTO to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminium hydroxide, calcium carbonate, magnesium hydroxide, magnesium aluminium silicate, synthetic aluminium silicate, synthetic hydrocalcite, magnesium aluminium hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Preferred cations include sodium, potassium, lithium, magnesium, calcium and ammonium. Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

Compounds of the invention may also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

The invention is now described with reference to the following examples which, are presented for the purpose of illustration only and are not limiting of the invention.

### EXAMPLE 1

### Topical TTO Formulations

Suitable topical vehicles for use in administration of TTO in accordance with this invention, and methods of preparation thereof, include the following. The term ‘qs’ refers to quantity specified. The numerical values represented reflect ratios of the various components.

1. Vanishing Creams:

(i) Cetomacrogol Cream

TTO qs

Cetomacrogol emulsifying wax 15

Liquid paraffin (by weight) 10

Chlorocresol 0.1

Propylene glycol 5

Distilled water to 100

Melt the cetomacrogol emulsifying wax with paraffin at about 70° C. Dissolve the chlorocresol and propylene glycol in about 50 parts of the distilled water warmed to about the same temperature. Mix, adjust to weight and stir until cool. Then add TTO in appropriate concentration, and mix thoroughly.

In a particularly preferred embodiment, to 100 g of cetomacrogol cream APF containing cetomacrogol emulsifying wax, liquid paraffin, chlorocresol, propylene glycol and water, add TTO to yield finished cream.

(ii) Aqueous Cream APF

TTO qs

Emulsifying ointment 30

Glycerol 5

Phenoxyethanol 1

Distilled water to 100

Melt the emulsifying ointment at about 70° C.

Dissolve the phenoxyethanol in the distilled water, warmed to about the same temperature. Mix, adjust to weight and stir until cool. Add TTO, stirring thoroughly.

(iii) Buffered Cream BPC 73

TTO qs

Citric acid 5

Sodium phosphate 25

Chlorocresol 1

Emulsifying ointment 300

Distilled water 669

Melt the emulsifying ointment with the aid of gentle heat, add the sodium phosphate, the citric acid and the chlorocresol, previously dissolved in the distilled water at the same temperature, and stir gently until cold. Add the TTO and mix very well.

2. Ointments:
(i) Emulsifying Ointment APF
   TTO qs
   Emulsifying wax 30
   White soft paraffin 50
   Liquid paraffin (by weight) 20
   Melt together and stir until cool. Add TTO in appropriate concentration in a portion of the base and then gradually incorporate the remainder, mixing thoroughly.
(ii) (ii) Ointment-as in Neomycin and Bacitracin
   Ointment BPC 73
   TTO qs
   Liquid paraffin
   White soft paraffin to 100
   Melt the white soft paraffin, incorporating the liquid paraffin, and stir until cold. Titurate the TTO with a portion of the base and gradually incorporate the remainder of the base.

3. Gels:
(i) Gel-as used in Lignocaine and

Chlorhexidine Gel APF

TTO qs

Tragacanth 2.5

Glycerol 25

Distilled water to 100

Mix the tragacanth with the glycerol and add most of the distilled water. Heat to boiling, cool, add TTO, adjust to weight and mix well. Protect finished product from light.

(ii) Vaginal gel B

Part A

Add 15.6 g Lutrol® F127 to 84.4 g deionised water, which is held at a temperature of 6° C. Combine with slow mixing to reduce air entrapment and place under vacuum for a few minutes to remove any trapped air after Lutrol® F127 is dissolved.

Part B

Add 0.20 g fumaric acid to 5.0 g alcohol by stirring until dissolved. Cool the solution to 10° C.

Part C

Combine 3.0 g TTO, 5.0 g propylene glycol and 2.0 g undecylenic acid and mix to dissolve all ingredients. Cool the solution to 10° C.

Gel Preparation

Place 84.8 g of the Lutrol® F127 solution of Part A in a vessel and hold at 10° C. Slowly add 5.2 g of the fumaric acid solution of Part B and mix well, maintaining the solution at 10° C. Slowly add 10.0 g of the TTO solution of Part C with gentle stirring and whilst maintaining the solution at 10° C. If necessary, remove any aeration by placing the gel under vacuum. Allow the gel to warm to room temperature.

(iii) Poloxamer gel 8C

Part A

Heat 76.3 g deionised water to 60-65° C., slowly add 16.7 g poloxamer 407 and stir gently for approximately 2 hours or until all the poloxamer is dissolved and the solution thickens. Allow the solution to cool to room temperature and leave overnight. Adjust the pH of the solution to 4.2-5.0 with potassium hydroxide.

Part B

Combine 3.0 g of PPG-26-Buteth-26/PEG-40 Hydrogenated castor oil oil, 3.0 g TTO and 1.0 g d-alpha tocopheryl acetate with gentle mixing.

Gel Preparation

Add 7.0 g of the TTO solution of Part B to 93.0 g of the room temperature poloxamer solution of Part A. Mix with gentle stirring until the solution thickens.

(iv) Poloxamer gel 8E

Part A

Heat 73.4 g deionised water to 60-65° C., slowly add 16.0 g poloxamer 407 and stir gently for approximately 2 hours or until all the poloxamer is dissolved and the solution thickens. Allow the solution to cool to room temperature and leave overnight. Adjust the pH of the solution to 4.2-5.0 with potassium hydroxide.

Part B

Combine 2.0 g Laureth-4, 1.0 g of Laureth-23, 6.0 g TTO and 1.0 g δ-alpha tocopheryl acetate, 0.1 g of 1.0 M (1 U/g) retinyl palmitate and 0.5 g panthenol. Heat solution to 40-45° C. with gentle mixing to dissolve all components.

Gel Preparation

Add 10.6 g of the TTO solution of Part B to 89.4 g of the room temperature poloxamer solution of Part A. Mix with gentle stirring until the solution thickens.

4. Sprays:

(i)-as used in Adrenaline and Atropine Spray BPC 73

TTO qs

Sodium metabisulphite 1Chlorbutol 5

Prophylene glycol 50

Distilled water to 1000

(ii)-as used in Indospray

TTO qs

Alcohol 95%

5. Lotions:

(i)-as used in Aminobenzoic Acid Lotion BPC 73

TTO qs

Glycerol 20

Alcohol 95% 60

Distilled water to 100

(ii)-Cetomacrogol Lotion APF

TTO qs

Cetomacrogol emulsifying wax 3

Liquid paraffin

Glycerol 10

Chlorhexidine gluconate solution 0.1

Distilled water to 100

Melt the cetomacrogol emulsifying wax with the liquid paraffin at about 60° C. and add, with rapid stirring, to the chlorhexidine solution previously diluted to 50 parts with distilled water at the same temperature. Mix, adjust to volume and stir until cold.

### EXAMPLE 2

### In Vivo Analyses of the Anti-Cancer Efficacy of Dilute Preparations of Tea Tree Oil (TTO) Applied Topically to Subcutaneous Murine Tumours (Mesothelioma and Melanoma)

PART A: Inhibitory and Regressive Effect of TTO on Murine Tumour Growth

The models used, murine AE17 mesothelioma and murine B16 melanoma tumour, both involve the implantation of tumour cell lines subcutaneously onto the rear flank of fully immuno-competent mice. Palpable tumours arise within 3-14 days. Tumour sizes can be measured using micro-callipers and tumour growth rates hence calculated.

Female fully immuno-competent C57BL/6J mice between 6-8 weeks of ages are obtained from the Animal Resources Centre (Perth, Australia) and maintained under SPF (specified pathogen free) housing conditions (Animal Care Unit, University of Western Australia).

AE17 cells were derived from the peritoneal cavity of C57BL/6J mice injected with asbestos fibres (Jackaman, 2003) and (Davis, 1992) and are injected subcutaneously at a concentration of 1×10⁷ per mouse (Needham, 2006). B16-F10 cells were obtained from ATCC, catalogue number: CRL-6322 and are injected subcutaneously at a concentration of 5×10⁵ per mouse

TTO obtained by the steam distillation of leaves of Melaleuca alternifolia was kindly provided by P. Guinane Pty. Ltd. Batches 1216 or A352 were used for all studies and had the composition as shown (Tables 1 and 2, compared to ISO 4730 ranges, Table 3) evaluated by gas-chromatography mass spectrometry carried out by NSW Department of Primary Industries, Diagnostic and Analytical Services, Environmental Laboratory, Wollongbar, NSW.

TABLE 1
Composition of Melaleuca alternifolia oil batch no. 1216

| Components | Percentage |
|---|---|
| 1. α-pinene | 2.4 |
| 2. sabinene | 0.3 |
| 3. α-terpinene | 9.0 |
| 4. limonene | 1.1 |
| 5. p-cymene | 3.1 |
| 6. 1,8-cineole | 3.7 |
| 7. γ-terpinene | 20.1 |
| 8. terpinolene | 3.2 |
| 9. terpinen-4-ol | 42.4 |
| 10. α-terpineol | 3.1 |
| 11. aromadendrene | 1.1 |
| 12. ledene | 0.6 |
| 13. δ-cadinene | 0.8 |
| 14. globlol | 0.2 |
| 15. viridiflorol | 0.1 |

TABLE 2
Composition of Melaleuca alternifolia oil batch no. A352

| Components | Percentage |
|---|---|
| 1. α-pinene | 2.2 |
| 2. sabinene | 2.0 |
| 3. α-terpinene | 10.0 |
| 4. limonene | 1.0 |
| 5. p-cymene | 1.4 |
| 6. 1,8-cineole | 3.6 |
| 7. γ-terpinene | 18.6 |
| 8. terpinolene | 3.5 |
| 9. terpinen-4-ol | 37.0 |
| 10. α-terpineol | 3.0 |
| 11. aromadendrene | 1.3 |
| 12. ledene | 0.9 |
| 13. δ-cadinene | 1.3 |
| 14. globlol | 0.3 |
| 15. viridiflorol | 0.1 |

TABLE 3
International standard ISO 4730 range for ‘Oil of Melaleuca-terpinen-4-ol
type”

| Components | Percentage |
|---|---|
| 1. α-pinene | 1-6 |
| 2. sabinene | Traces-3.5 |
| 3. α-terpinene | 5-13 |
| 4. limonene | 0.5-1.5 |
| 5. p-cymene | 0.5-8 |
| 6. 1,8-cineole | Traces-15 |
| 7. γ-terpinene | 10-28 |
| 8. terpinolene | 1.5-5 |
| 9. terpinen-4-ol | 30-48 |
| 10. α-terpineol | 1.5-8 |
| 11. aromadendrene | Traces-3 |
| 12. ledene | Traces-3 |
| 13. δ-cadinene | Traces-3 |
| 14. globlol | Traces-1 |
| 15. viridiflorol | Traces-1 |

DMSO (Hybri Max®) was purchased from Sigma Aldrich Ltd Catalogue number D2650.

Novasel® Gel had the composition as shown in Table 4. Novasel® Gel was provided by Novasel Australia Pty Ltd.

TABLE 4
Composition of Novasel ® 3% TTO Gel

| Components | Percentage |
|---|---|
| Poloxamer 407 | 15.74 |
| Water Purified | 77.01 |
| PPG-26-Buteth-26 (and) | 3.00 |
| PEG-40 Hydrogenated castor oil | |
| Melaleuca oil | 3.00 |
| Tocopheryl acetate (VitE) | 1.00 |
| Lactic acid | 0.25 |

(i) The Effect of Daily, Topical Treatment with 10% TTO in DMSO on the Growth of Established Subcutaneous AE17 Mesothelioma and B 16 Melanoma Tumours

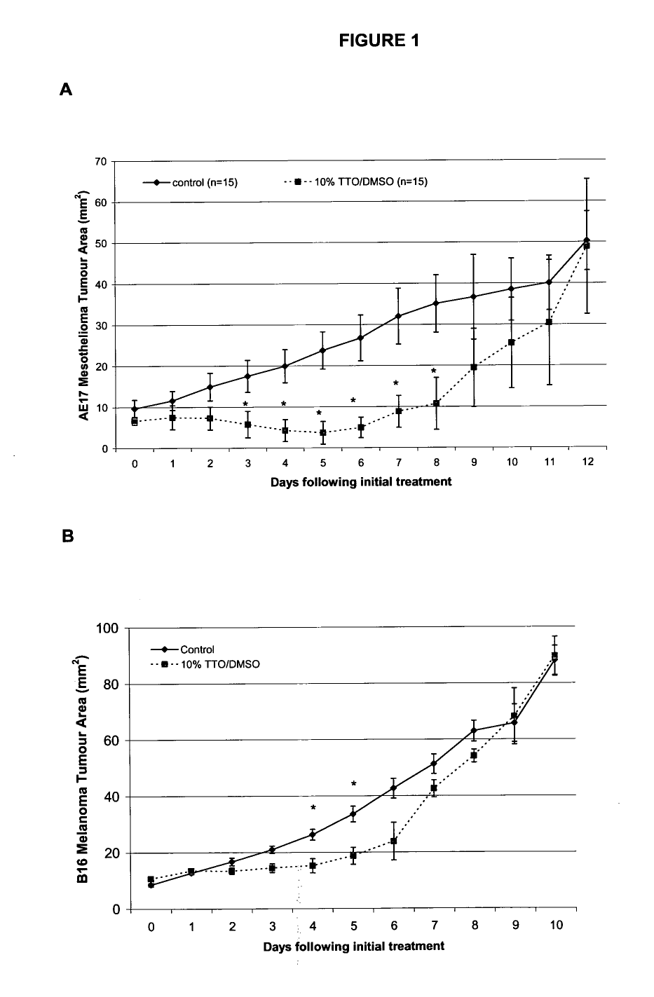

Mice with established (˜9 mm²) AE17 (FIG. 1A) and B16 (9 mm²) (FIG. 1B) subcutaneous tumours, were treated topically with 50 μl 10% TTO in DMSO or DMSO alone (solvent control) for 4 days.

Following 3 treatments with 10% TTO/DMSO, mesothelioma tumour area was significantly (P<0.05) reduced (FIG. 1). This period of tumour growth inhibition extended for approximately 6 days and included in some cases a period of complete tumour regression. Upon cessation of treatment, tumours relapsed to normal tumour growth but with some degree of variability (2 out of 15 mice displayed no signs of relapse until 3 months post treatment).

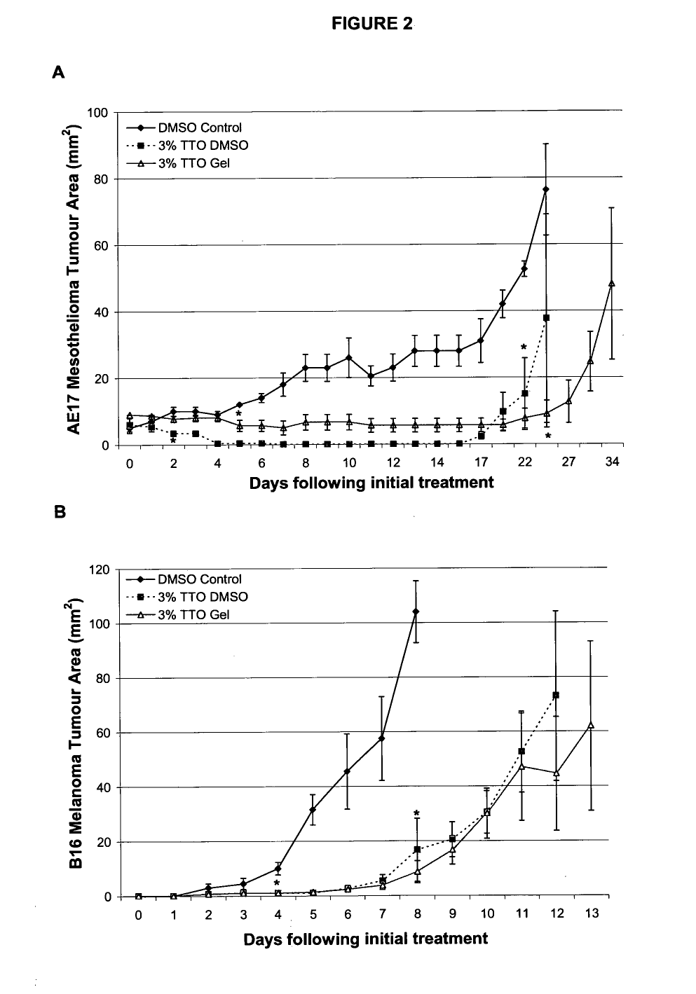

Topical 10% TTO/DMSO was also found to significantly (P<0.05) retard the growth of established (˜9 mm²) B16-F10 melanomas (FIG. 2). Tumours resumed growth upon cessation of treatment, and grew rapidly to 100 mm². Side effects of four daily topical treatments with 10% TTO/DMSO in both tumour models manifested as skin irritation; dryness, erythema, edema, with eschar formation which, began to heal 3 days post cessation of treatment and completely resolved.

(ii) The Effect of Daily, Topical Treatment with 3% TTO in DMSO Compared to 3% TTO Novasel® gel on the Growth of Established Subcutaneous AE17 Mesothelioma and Non-Established Subcutaneous B16 Melanoma Tumours

Mice with subcutaneous tumours were treated topically with a) 50 μl DMSO (solvent control), b) 50 μl 3% TTO Novasel® Gel and c) 50 μl 3% TTO/DMSO for 16 days (AE17 mesothelioma, FIG. 2A) and 13 days (B16 melanoma, FIG. 2B).

It was found that the 3% TTO gel inhibited mesothelioma (AE17) tumour growth but did not reduce tumour area (FIG. 2A). No side effects were evident and skin remained normal. Upon cessation of treatment, tumours resumed growth but remained slow growing with a tumour area of 9 mm² compared to control tumours of 77 mm² at day 25 (2 out of 3 mice). 3% TTO in DMSO completely inhibited mesothelioma (AE17) tumour growth and induced significant tumour regression following just one treatment (FIG. 2A). It was shown that ˜100% tumour regression was induced by day 4 (6 out of 6 mice). Side effects manifested as some skin dryness that healed completely following cessation of treatment. Tumour growth resumed in 5 out of 6 mice but by day 22 (6 days post treatment) the tumours were still significantly smaller (15 mm²) than those of control mice (52 mm²) mice. A tumour failed to relapse in 1 mouse, and the mouse remained tumour free for 3 months.

3% Novasel® TTO gel and 3% TTO in DMSO also inhibited B16 melanoma tumour growth (FIG. 2B), but did not induce tumour regression. Treatment with the 3% Novasel® TTO gel results in no skin side effects, however treatment with 3% TTO in DMSO resulted in some skin dryness. Following 6 days of treatment, the tumours began to grow; but remained significantly slower growing (8-16 mm², 9 days post tumour inoculation) compared with control DMSO alone treated tumours which reached ˜100 mm² 9 days post tumour inoculation (treated tumours reached ˜90 mm² in ⅔ mice 14 days post tumour inoculation).

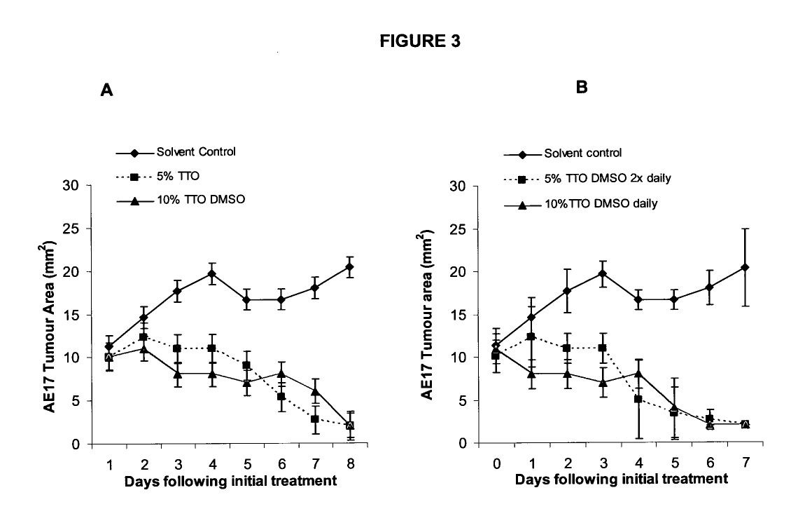

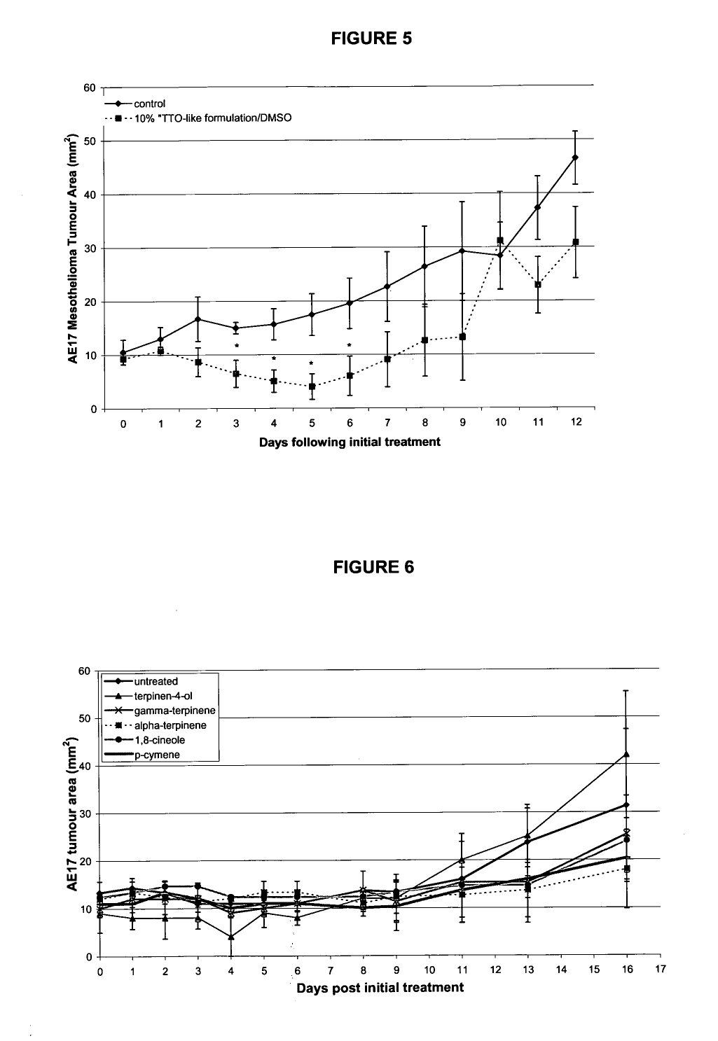

(iii) The Effect of twice daily topical treatment with 5% TTO in DMSO compared to 10% TTO in DMSO Administered Daily (FIG. 3A) and 5% TTO in DMSO Twice Daily Compared with 10% TTO in DMSO Every Second Day (FIG. 3B) Against Established Subcutaneous AE17 Mesotheliomas.

5% TTO in DMSO administered once daily was compared to 10% TTO in DMSO every second day in an attempt to reduce the skin irritation (FIG. 3A). A total of four 10% TTO treatments and a total of eight TTO treatments of 5% daily were applied and limited due to apparent skin irritation. Interestingly both treatment regimes yielded similar tumour regression.

In contrast, 5% TTO in DMSO was administered twice daily and compared to 10% TTO in DMSO administered daily (FIG. 3B) in an attempt to treat mice with the higher more effective doses of TTO in DMSO but in a regime that may reduce skin irritation. Similarly, both 5% TTO and 10% TTO administered twice daily and every second day respectively induced tumour regression, but again were limited to a total of 4 doses of 10% TTO and 8 doses of 5% TTO due to skin irritation in the mice (FIG. 3B).

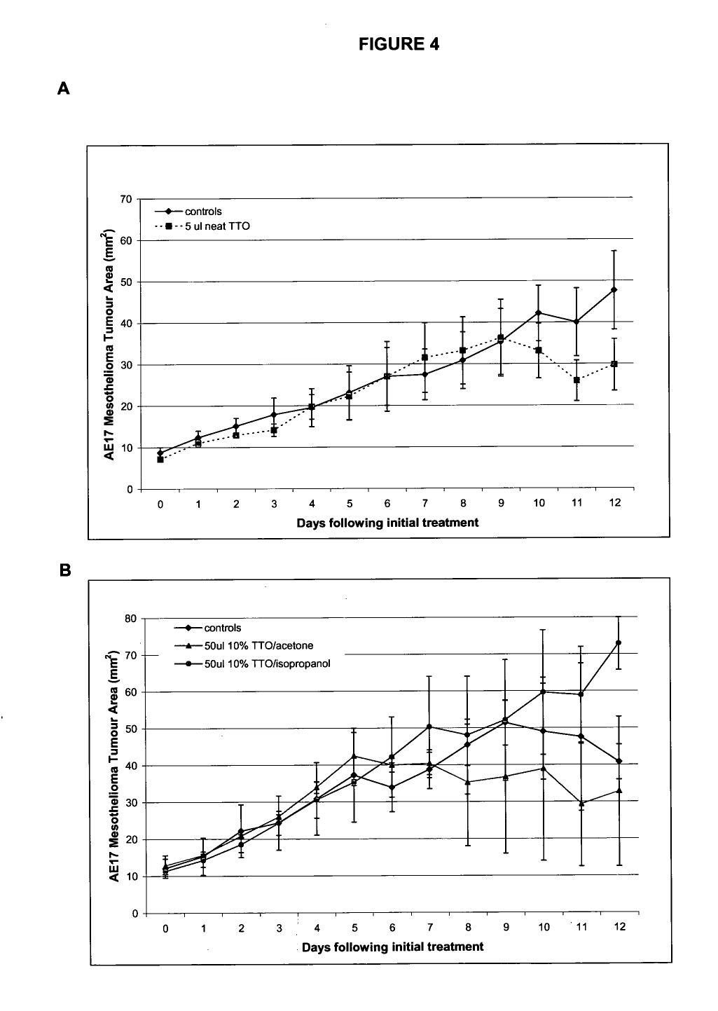

(iv) The Effect of 5 μl Topical Neat TTO Treatment, 50 μl 10% TTO in Isopropanol or 10% TTO in Acetone and 5 Major Components of TTO (FIGS. 4A and 4B, FIG. 5, FIG. 6).

In order to establish the importance of a vehicle for TTO's antitumour response, we subsequently examined the efficacy of 5 μl topical neat TTO to established AE17 subcutaneous tumours (FIGS. 4A, B). Treatment with neat TTO resulted in only mild skin irritation, thus allowing up to 7 daily treatments to be applied. However, even with the increased application time, the dose of neat TTO equivalent to that found in 10% TTO in DMSO had no growth inhibitory effects (FIG. 4A). This suggests a vehicle is required for TTO's antitumour response, by enhanced penetration through the skin, and/or by decreased evaporation of the TTO components from the skin.

In addition, mice with established AE17 subcutaneous tumours were treated topically with 50 μl 10% TTO in isopropanol or 10% TTO in acetone and compared to controls to determine if either of these vehicles afforded TTO antitumour efficacy (FIG. 4B). The 10% TTO in isopropanol or acetone had no inhibitory effect on tumour growth (FIG. 4B). Side effects of 10% TTO/isopropanol or 10% TTO/acetone treatment manifested as some dryness and erythema with skin healing upon cessation of treatment. Following both these treatments, skin reactions were not as severe as post treatment with 10% TTO in DMSO thus allowing up to 7 daily topical treatments. It was clear from these experiments, that DMSO is beneficial for the antitumour efficacy of TTO.

In order to establish if the antitumour efficacy of TTO could be attributed to the 5 major components which make up 80% of the oil, a TTO-like formulation was made by mixing the following major components of TTO at concentrations equivalent to their composition in ISO standard TTO; 40% terpinen-4-ol, 20% γ-terpinene, 10% α-terpinene 5% 1,8-cineole and 5% p-cymene, 20% Ethanol (EtOH) was included to total the solution composition to 100%. We examined the efficacy of 10% topical TTO-like formulation in established AE17 subcutaneous tumours (FIG. 5). The TTO-like formulation induced a significant period of tumour growth inhibition between days 3 and 6 compared with DMSO controls and a day of significant tumour regression 1 day post TTO-like treatment (Day 5: tumour size of 4 mm² compared with initial tumour size of 9.3 mm², P<0.05). The TTO-like formulation induced similar skin irritation as with 10% TTO in DMSO.

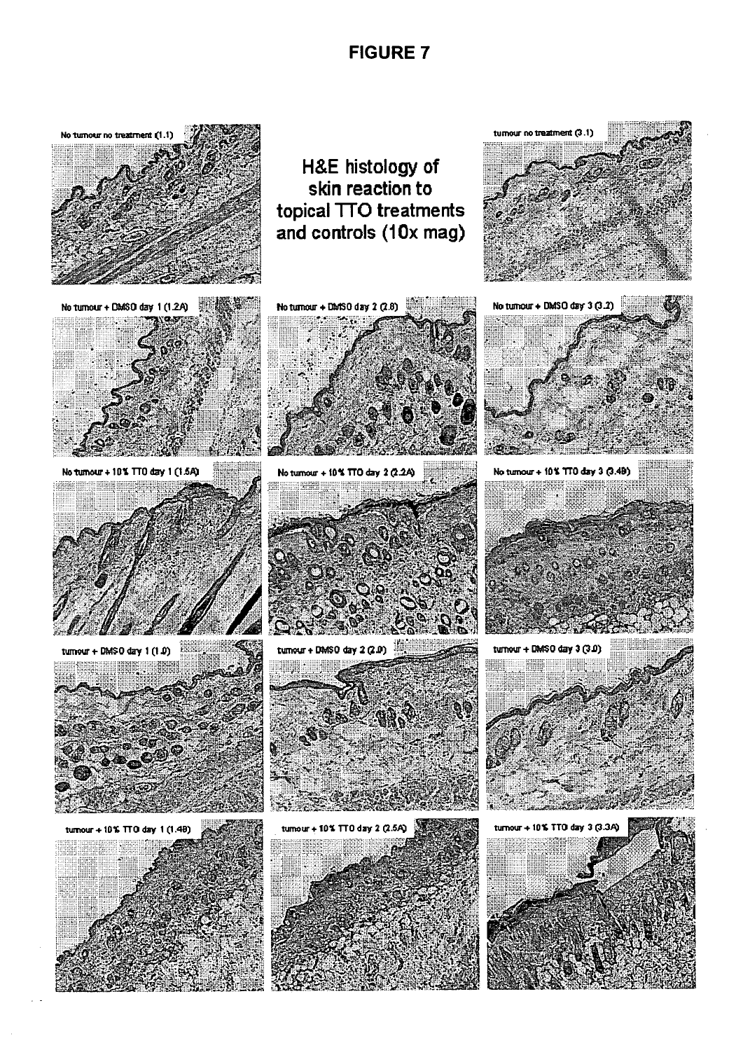

We also examined the efficacy of the individual major components of TTO at concentrations equivalent to their composition in ISO standard of TTO (FIG. 6). Individually, TTO major components had no effect of AE17 tumour growth suggesting the combination of the five major components of TTO is beneficial in TTO's anti-tumour efficacy.

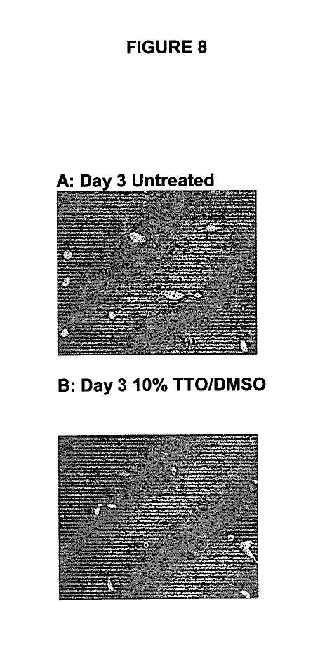

Microscopic analyses by H&E histology (FIG. 7) found that epidermal skin irritation was only seen as a result of 10% TTO in DMSO treatment with increased numbers of neutrophils present. Dermal inflammation was also seen in 10% TTO in DMSO treated skin/tumour sections and the inflammation was again characterized by an abundance of neutrophils. Dermal inflammation increased with subsequent 10% TTO in DMSO treatments with diverse immune cells present including macrophages, mast cells and lymphocytes, but not eosinophils. FIG. 7 shows H&E staining of histology sections through the skin of C57BL/6J mice bearing AE17 mesothelioma tumours (A) Day 0, prior to treatment (B) 1 day post treatment with 90% DMSO alone (C) 1 day post treatment with 10% TTO in DMSO (D) post 3 treatment with 90% DMSO (E) post 3 treatments with 10% TTO in DMSO.

We also examined 10% TTO in DMSO treated animals for signs of systemic toxicity resulting from topical application. Liver H&E histology showed no obvious signs of liver toxicity (FIGS. 8A, B) between untreated control mice and 10% TTO in DMSO treated mice. This was further confirmed by serum samples from these mice analysed for liver toxicity by liver enzyme analysis including ALT, AST and GGT which showed no difference between control, untreated mice compared to 10% TTO in DMSO treated mice (data not shown).

These experiments demonstrate that TTO applied topically to subcutaneous murine mesothelioma or melanoma tumours can induce tumour growth inhibition and AE17 tumour regression, with only short-term local skin inflammation, which rapidly heals with no detectable systemic toxicity. The increase in neutrophils numbers in the skin of mice following 10% TTO in DMSO treatment indicates a possible antitumour immune cell involvement.

### REFERENCES

The following references are included for ease of reference only and are merely included to facilitate an understanding of the present invention. The inclusion of these references is not an acknowledgement or admission that any of the material referred to herein, or the matter contained in these references, is (or was), part of the common general knowledge as at the priority date of the application.
Carson, C. F., Hammer, K. A. and Riley, T. V. (1996) “In-vitro activity of the essential oil of Melaleuca alternifolia against Streptococcus spp”, J Antimicrob Chemother, 37, 1177-1178.
Carson, C. F., Hammer, K. A. and Riley, T. V. (2006) “Melaleuca alternifolia (Tea Tree) oil: a review of antimicrobial and other medicinal properties”, Clin Microbiol Rev, 19, 50-62.
Challacombe, J. M., Suhrbier, A., Parsons, P. G., Jones, B., Hampson, P., Kavanagh, D., Rainger, G. E., Morris, M., Lord, J. M., Le, T.T ., Hoang-Le, D. and Ogbourne, S. M. (2006) “Neutrophils are a key component of the antitumor efficacy of topical chemotherapy with ingenol-3-angelate”, J lmmunol, 177, 8123-8132.
Davis, M. R., Manning, L. S., Whitaker, D., Garlepp, M. J. and Robinson, B. W. (1992) “Establishment of a murine model of malignant mesothelioma”, Int J Cancer, 52, 881-886.
Geller, A. C., Swetter, S. M., Brooks, K., Demierre, M. F. and Yaroch, A. L. (2007) “Screening, early detection, and trends for melanoma: current status (2000-2006) and future directions”, J Am Acad Dermatol, 57, 555-572; quiz 573-556.
Jackaman, C., Bundell, C. S., Kinnear, B. F., Smith, A. M., Filion, P., Van Hagen, D., Robinson, B. W. and Nelson, D. J. (2003) “IL-2 intratumoral immunotherapy enhances CD8+ T cells that mediate destruction of tumor cells and tumor-associated vasculature: a novel mechanism for IL-2”, J Immunol, 171, 5051-5063.
Lee, J. R., Shin, J. H., Park, J. H., Song, S. U. and Choi, G. S. (2007) “Combined treatment with intratumoral injection of dendritic cells and topical application of imiquimod for murine melanoma”, Clinical and Experimental Dermatology, 32, 541-549.
Needham, D. J., Lee, J. X. and Beilharz, M. W. (2006) “Intra-tumoural regulatory T cells: a potential new target in cancer immunotherapy”, Biochem Biophys Res Commun, 343, 684-691.
Ogbourne, S. M., Suhrbier, A., Jones, B., Cozzi, S. J., Boyle, G. M., Morris, M., Mcalpine, D., Johns, J., Scott, T. M., Sutherland, K. P., Gardner, J. M., Le, T. T., Lenarczyk, A., Aylward, J. H. and Parsons, P. G. (2004) “Antitumor activity of 3-ingenyl angelate: plasma membrane and mitochondrial disruption and necrotic cell death”, Cancer Res, 64, 2833-2839.
Rabinovich, G. A., Gabrilovich, D. and Sotomayor, E. M. (2007) “Immunosuppressive strategies that are mediated by tumor cells”, Annu Rev Immunol, 25, 267-296.
Standardisation, I. O. F. (2004) “ISO 4730:2004: Oil of Melaleuca, Terpinen-4-ol type (Tea Tree Oil)”, International Organisation for Standardisation, Geneva Switzerland

1. A composition for use in the treatment and/or prevention of cancer, wherein the composition comprises a therapeutically effective amount of TTO.

2.-4. (canceled)

5. A composition according to claim 1, wherein the composition comprises TTO at a concentration selected from the group consisting of; between 0.1% and 80% TTO by weight of the composition; between 1% and 30% TTO by weight of the composition; and between 3% and 15% TTO by weight of the composition.

6. A composition according to claim 1, wherein the composition comprises a percentage of TTO by weight of the composition wherein the percentage is selected from the group consisting of: 15%, 14%, 13%, 12%, 11%, 10%, 9%, 8%, 7%, 6%, 5%, 4% and 3%.

7.-9. (canceled)

10. A composition according to claim 1, wherein the composition comprises a pharmaceutically or therapeutically acceptable carrier and/or diluent.

11. (canceled)

12. A composition according to claim 1, wherein the composition is adapted for intravenous, intra-arterial, intraperitonial, intramuscular, subcutaneous, intranasal, topical or transdermal administration.

13.-19. (canceled)

20. A composition for the treatment and/or prevention of cancer, wherein the composition comprises a therapeutically effective amount of a compound selected from the group consisting of; α-pinene, sabinene, α-terpinene, limonene, p-cymene, 1,8-cineole γ-terpinene, terpinole, terpinen-4-ol, α-terpineol, aromadendrene, ledene, δ-cadinene, globlol and viridiflorol.

21.-22. (canceled)

23. A method of treating and/or preventing cancer, said method comprising the step of administering to a subject in need thereof a composition according to claim 1.

24. (canceled)

25. A method according to claim 23, wherein the cancer is selected from the group consisting of sarcomas, carcinomas and other solid tumour cancers, tumours of the central nervous system, breast cancer, prostate cancer, skin cancer, cervical cancer, uterine cancer, lung cancer, ovarian cancer, testicular cancer, thyroid cancer, astrocytoma, glioma, pancreatic cancer, stomach cancer, liver cancer, colon cancer, renal cancer, bladder cancer, oesophageal cancer, cancer of the larynx, cancer of the parotid, cancer of the biliary tract, rectal cancer, endometrial cancer, adenocarcinomas, small cell carcinomas, neuroblastomas, mesotheliomas, adrenocortical carcinomas, epithelial carcinomas, desmoid tumours, desmoplastic small round cell tumours, endocrine tumours, Ewing sarcoma family tumours, germ cell tumours, hepatoblastomas, hepatocellular carcinomas, lymphomas, non-rhabdomyosarcome soft tissue sarcomas, osteosarcomas, peripheral primative neuroectoderrnal tumours, retinoblastomas, rhabdomyosarcomas and Wilms tumours.

26. A method according to claim 23, wherein the cancer is skin cancer.

27. A method according to claim 23, wherein the cancer is basal cell carcinoma cell carcinoma, squamous cell carcinoma or melanoma.

28. (canceled)

29. A method according to claim 23, wherein the composition is administered intravenously, intra-arterially, intraperitoneally, intramuscularly, subcutaneously, intranasally or transdermally.

30. (canceled)

31. A method according to claim 23, wherein the composition is administered topically.

32.-33. (canceled)

34. A dosage form comprising a therapeutically effective amount of a composition according to claim 1, stored in a vial or container, wherein the vial or container is labelled with instructions that the composition is administered to the subject for the treatment or prevention of cancer.

35.-48. (canceled)
